(19) United States
(12) Patent Application Publication
(10) Pub. No.: US 2004/0162273 A1
(43) Pub. Date: Aug. 19, 2004

(54) Powder pharmaceutical compositions

(75) Inventors: Liza Elaine Achong, Miranda (VE); Oswaldo David Romero, Caracas (VE)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 10/349,665

(22) Filed: Jan. 23, 2003

(51) Int. Cl. A61K 31/60; A61K 31/192; A61K 31/16; A61K 31/137

(52) U.S. Cl. 514/165; 514/649; 514/570; 514/569; 514/629

(57) ABSTRACT

The present invention is directed to powder pharmaceutical compositions which are dissolvable in a liquid such as hot water, wherein the compositions provide for a therapeutic effect in the treatment of symptoms associated with the common cold and/or flu. These powder pharmaceutical compositions comprise a select pharmaceutical active that is especially effective in alleviating cold and/or flu symptoms.

### FIELD OF THE INVENTION

The present invention relates to powder pharmaceutical compositions which are highly effective in the treatment of symptoms related to the common cold and influenza. In particular, the present invention relates to powder pharmaceutical compositions which contain pharmaceutical actives that are highly efficacious in inhibiting and/or treating cold and/or influenza like symptoms, wherein the powder pharmaceutical compositions are dissolvable in a liquid such as hot water prior to ingestion for relief of these symptoms.

### BACKGROUND OF THE INVENTION

Symptoms of the common cold and/or flu (i.e. influenza) typically include coughing, sneezing, headaches, congestion, sore throat, stuffy nose, runny nose, fever, and the like. Pharmaceutical products to treat such symptoms associated with the common cold and/or flu can be categorized as liquid elixirs, cough syrups, cold and flu capsules, cold and flu tablets, effervescent tablets, mouth and nasal sprays, cough drops, and so forth. These pharmaceutical products have been shown to effectively inhibit and/or treat symptoms of the common cold and/or flu.

The most commonly employed cold and/or flu treating pharmaceutical products are ingested or bucally administered to inhibit and/or treat onsets or fully developed cold and/or flu symptoms. The pharmaceutical products typically contain one or more pharmaceutical actives dissolved or dispersed in a carrier system for ingestion or bucal delivery into the bloodstream. For example, pharmaceutical products sold under the Nyquil®, Dayquil®, and Vicks®, tradenames are especially consumer preferable drops, liquids and capsules formulations that are highly effective in inhibiting and/or treating cold and/or flu symptoms.

Although most consumers prefer cold and flu pharmaceutical products in the form of cough drops, liquids, or capsules, marketable cold and flu pharmaceutical products in the form of powders and effervescent tablets have met consumer needs in combating the cold and/or flu. Commonly known powder and effervescent tablet products include those pharmaceutical products sold under the TheraFlu® and Alka Seltzer Plus® tradenames. Pharmaceutical products sold under the TheraFlu® tradename have been shown to comprise a combination of multiple pharmaceutical active ingredients to treat symptoms of the common cold and/or flu, specifically a combination of acetaminophen, pseudoephedrine hydrochloride, chlorpheniramine maleate, and dextromethorphan hydrobromide active ingredients.

In addition to TheraFlu® and other commercially sold powder and effervescent tablet pharmaceutical products that are suitable for the inhibition and/or treatment of cold and/or flu symptoms, there exist prior disclosures of water-soluble powder and effervescent formulations. For example, CA 2084028 describes an effervescent composition that is dissolved in hot water prior to ingestion for the treatment of cold and flu symptoms. The published document EP 418,564 discloses a water-soluble effervescent cold or sinus allergy medicine composition that can be used in powder-granulated form and that has a reduced sodium content.

In spite of commercial products and prior disclosures of water-soluble cold and/or flu compositions that are manufactured in the form of powders and tablets, the need still exists for a cold and/or flu composition such as a non-effervescent, water-soluble powder that is highly effective in the inhibition and/or treatment of cold and/or flu symptoms. It has been found that cold and/or flu compositions can be formulated to contain one or more specific pharmaceutical actives to alleviate symptoms associated with the common cold and/or flu. These compositions are formulated into powders that are especially soluble in a liquid such as hot water.

### SUMMARY OF THE INVENTION

The present invention is directed to powder pharmaceutical compositions which comprise a phenylephrine pharmaceutical active, wherein the compositions are preferably dissolved in a hot liquid prior to ingestion.

It has been found that powder pharmaceutical compositions, particularly powder pharmaceutical compositions that contain a combination of phenylephrine and acetaminophen active ingredients, are especially effective in treating symptoms associated with the common cold and/or flu. The powder pharmaceutical compositions of the present invention can also be formulated to contain aesthetically pleasing flavor and sweetener ingredients.

### DETAILED DESCRIPTION OF THE INVENTION

The powder pharmaceutical compositions of the present invention are preferably dissolved in a hot liquid prior to ingestion for the relief of symptoms associated with the common cold and/or flu. These compositions comprise one or more select pharmaceutical actives, especially a select combination of analgesic and decongestant pharmaceutical actives for the inhibition and/or treatment of cold and/or flu symptoms.

The terms “dissolved” or “dissolvable” as used herein refer to the solubilization of the powder pharmaceutical compositions in liquids such as cold and hot water. The powder pharmaceutical compositions of the present invention are considered soluble in a liquid upon mixing the powder with the liquid until the formation of a clear, translucent, or transparent solution.

The term “liquid” as used herein refers to water-like or semi-solid substances that are free flowing, and that can form a solution with the powder pharmaceutical compositions defined herein such that the powder pharmaceutical composition is heterogeneously or homogeneously mixed into a solution prior to ingestion for the delivery of an effective dosage of the pharmaceutical actives defined herein.

The term “effective dosage” as used herein refers to a concentration of any pharmaceutical active defined herein that is ingested, including ingestion by bucal administration, to provide for the intended relief of symptoms associated with the common cold and/or flu. The “effective dosage” can vary dependant on the pharmaceutical active ingested, however, effective dosages can typically range from about 1 milligram (mg) to about 1000 milligram (mgs) per powder pharmaceutical composition.

The powder pharmaceutical compositions of the present invention can comprise, consist of, or consist essentially of the elements and limitations of the invention described herein, as well as any of the additional or optional ingredients, components, or limitations described herein.

All percentages, parts and ratios are by weight of the powder pharmaceutical compositions, unless otherwise specified. All such weights as they pertain to listed ingredients are based on the specific ingredient level and, therefore, do not include carriers or by-products that may be included in commercially available materials, unless otherwise specified.

### Pharmaceutical Active

The powder pharmaceutical compositions of the present invention comprise a pharmaceutical active that provides a safe and effective amount of relief from symptoms associated with the common cold and/or flu. The pharmaceutical active can be included in the composition as an individual active compound or as a combination of active ingredients, provided that the pharmaceutical active provides for the desired therapeutic effect on the body when the powder pharmaceutical compositions are administered after the compositions have been dissolved in a liquid defined hereinbelow. Typically, the powder pharmaceutical compositions are admixed with liquid prior to ingestion such that an effective dosage of the pharmaceutical active is administered in a safe and effective amount. As used herein, the term “safe and effective amount” refers to an amount with provides a therapeutic benefit with minimal or no adverse reactions.

The pharmaceutical active suitable for use herein is typically used to treat symptoms associated with cold and/or flu respiratory ailments including those symptoms of a stuffy or runny nose, soreness and inflammation in the nose, soreness and inflammation in the throat, fits of coughing, general aches in the body, fever, headache, sneezing, and so forth. For effective treatment of cold and/or flu symptoms, the powder pharmaceutical compositions of the present invention comprise the pharmaceutical active at concentrations ranging from about 0.01% to about 20%, preferably from about 0.02% to about 13%, more preferably from about 0.05% to about 10%, by weight of the composition.

Nonlimiting examples of a pharmaceutical active suitable for use herein include those active compounds, and their pharmaceutically-acceptable salts, which have a molecular weight of less than 500 grams per mole and which fall into at least one of the following pharmacological classifications: antitussives, antihistamines, non-sedating antihistamines, decongestants, expectorants, mucolytics, analgesics, antipyretics, anti-inflammatory agents, and mixtures thereof. References that describe the use of such actives include J. G. Hardman, The Pharmacologic Basis of Therapeutics, Ninth Edition, McGraw-Hill, New York, 1995, the description of which is incorporated by reference herein.

Nonlimiting examples of antitussives suitable for use as a pharmaceutical active herein include those antitussive compounds which are especially effective in arresting uncontrollable fits of coughing, specific nonlimiting examples of which include codeine, dextromethorphan, dextrorphan, hydrocodone, noscapine, oxycodone, pentoxyverine, and mixtures thereof. Dextromethorphan is the most preferred antitussive suitable for use as a pharmaceutical active herein, and is further described in U.S. Pat. No. 5,196,436, issued to Smith on Mar. 23, 1993, which description is incorporated by reference herein. As used herein, “dextromethorphan” means racemethorphan, (±)-3-Methoxy-17-methylmorphinan, dl-cis-1,3,4,9,10,10a-hexahydro-6-methoxy-11-methyl-2H-10,4a-iminoethanophenanthrene, and pharmaceutically-acceptable salts thereof including dextromethorphan hydrobromide.

Nonlimiting examples of antihistamines suitable for use as a pharmaceutical active herein include acrivastine, azatadine, brompheniramine, brompheniramine maleate, chlorpheniramine, chlorpheniramine maleate, clemastine, cyproheptadine, dexbrompheniramine, dimenhydrinate, diphenhydramine, diphenhydramine hydrochloride, doxylamine, doxylamine succinate, hydroxyzine, meclizine, pheninamine, phenyltoloxamine, promethazine, pyrilamine, pyrilamine maleate, tripelennamine, triprolidine, and mixtures thereof.

Nonlimiting examples of non-sedating antihistamines suitable for use as a pharmaceutical active herein include astemizole, cetirizine, ebastine, fexofenadine, loratidine, terfenadine, and mixtures thereof.

Nonlimiting examples of decongestants suitable for use as a pharmaceutical active herein include phenylpropanolamine, pseudoephedrine, pseudoephedrine hydrochloride, pseudoephedrine sulfate, ephedrine, phenylephrine, phenylephrine hydrochloride, oxymetazoline, and mixtures thereof.

Nonlimiting examples of expectorants suitable for use as a pharmaceutical active herein include ammonium chloride, guafenesin, ipecac fluid extract, potassium iodide, and mixtures thereof.

Nonlimiting examples of mucolytics suitable for use as a pharmaceutical active herein include acetylcycsteine, ambroxol, bromhexine, and mixtures thereof.

Nonlimiting examples of analgesics, antipyretics, and anti-inflammatory agents suitable for use as a pharmaceutical active herein include acetaminophen, aspirin, sodium salicylate, salicylamide, diclofenac, diflunisal, etodolac, fenoprofen, flurbiprofen, ibuprofen, ketoprofen, ketorolac, nabumetone, naproxen, piroxicam, caffeine, and mixtures thereof.

Preferably, the powder pharmaceutical compositions of the present invention comprise a phenylephrine pharmaceutical active, particularly phenylephrine hydrochloride. Most preferably, the powder pharmaceutical compositions of the present invention comprise a combination of phenylephrine hydrochloride and acetaminophen as the pharmaceutical active. It has been found that powder pharmaceutical compositions can be formulated to contain this select combination of pharmaceutical active to result in highly effective relief of cold and/or flu symptoms. When the powder pharmaceutical compositions of the present invention comprise this select combination of pharmaceutical active, the concentration of the phenylephrine hydrochloride ranges from about 0.05% to about 0.5%, preferably from about 0.08% to about 0.4%, more preferably from about 0.1% to about 0.3%, by weight of the composition; wherein the concentration of acetaminophen ranges from about 1% to about 20%, preferably from about 4% to about 15%, more preferably from about 6% to about 13%, by weight of the composition.

### Dissolvable Liquid

The powder pharmaceutical compositions of the present invention are dissolvable in a liquid to form a solution prior to ingestion of the composition to treat symptoms of the common cold and/or flu. The amount of liquid that can be used to dissolve the powder pharmaceutical compositions will vary dependant on factors such as the composition to liquid ratio, the compositional ingredients, the type of liquid, and so forth. Typically, from about 4 grams to about 20 grams of a powder pharmaceutical composition is dissolved in from about 5 ounces to about 8 ounces of liquid to form a solution that is ingested to treat symptoms of the common cold and/or flu.

The liquid suitable for use to dissolve the powder pharmaceutical compositions herein is generally a liquid at ambient temperature. The liquid can be water-like or a semisolid liquid material under ambient conditions, provided that the liquid can dissolve the powder pharmaceutical composition to form an ingestible solution. The terms “ambient temperature and “ambient conditions” are used interchangeably herein to refer to surrounding conditions at about one atmosphere of pressure, at about 50% relative humidity, at about 25° C.

The liquid suitable for use herein is preferably a hot liquid, however, the powder pharmaceutical compositions can be dissolved in cold liquids. Nonlimiting examples of preferred hot liquid materials include hot water, hot tea, hot milk, hot apple cider, and mixtures thereof. Nonlimiting examples of suitable cold liquid materials include cold water, ice tea, orange juice, grape juice, apple juice, grapefruit juice, cranberry juice, pineapple juice, and mixtures thereof.

### Optional Components

The powder pharmaceutical compositions of the present invention may further comprise one or more optional components known or otherwise effective for use in pharmaceutical compositions, provided that the optional components are physically and chemically compatible with the pharmaceutical active described hereinabove, or do not otherwise unduly impair product stability, aesthetics, or performance. The optional components can be included in the powder pharmaceutical compositions at concentrations ranging from about 0.001% to about 89.8%, preferably from about 0.01% to about 80%, by weight of the composition. Such optional components include materials such as flavoring agents, sweeteners, dyes, antioxidants including citric acid, ascorbic acid, preservatives, and the like. Nonlimiting examples of suitable optional flavoring agents include materials such as anise, oil of peppermint, oil of clove, eucalyptus, lemon, lime, honey, honey lemon, red fruit, mint, grapefruit, orange, grape, cherry, cherry cola, berry, and mixtures thereof. Nonlimiting examples of suitable optional sweeteners include materials such as aspartame, saccharin and its salts including calcium saccharin and sodium saccharin, natural sugars, and mixtures thereof.

### Method of Treatment

The powder pharmaceutical compositions of the present invention are dissolvable in a liquid such as hot water for treatment of symptoms associated with the common cold and/or flu. These compositions have been found to be especially effective in treating cold and/or flu symptoms at composition dosages ranging from about 4 grams to about 20 grams per powder pharmaceutical composition. It is believed that the pharmaceutical active component of the powder pharmaceutical compositions of the present invention provides for effective dosages in the relief of cold and/or flu symptoms, wherein the effective dosages can vary dependant on the pharmaceutical active included in the selected powder pharmaceutical composition.

Generally, the powder pharmaceutical compositions can be administered at the composition dosages specified herein to result in the administration of the pharmaceutical active at effective dosages ranging from about 1 mg to about 1000 mgs. By way of example, a typical composition dosage of the powder pharmaceutical compositions of the present invention are prepared to comprise a select pharmaceutical active that provides for a therapeutic effect in the relief of cold and/or flu symptoms, wherein the select pharmaceutical active comprise a combination of phenylepherine hydrochloride and acetaminophen at phenylepherine hydrochloride dosage ranging from about 9 mgs to about 11 mgs and acetaminophen dosage ranging from about 450 mgs to about 550 mgs per powder pharmaceutical composition.

The powder pharmaceutical compositions defined herein can be administered as desired or necessary for the relief of cold and/or flu symptoms, however, the powder pharmaceutical compositions are typically administered from one to six times per day (e.g., one to six times in a 24 hour period). For example, for individuals between the ages of 12 to 18 years old, the powder pharmaceutical compositions of the present invention are typically administered once every 6 hours and no more than four times in a 24 hour period. For adults over 18 years old, the compositions are typically administered once every 4 hours and no more than six times in a 24 hour period. The powder pharmaceutical compositions of the present invention are preferably not administered to children under 12 years old. The powder pharmaceutical compositions of the present invention can be administered less than the typical frequency of one to six times per day dependant on the severity of the symptoms, patient compliance, and so forth, however, the compositions are preferably not administered for more than ten consecutive days. It has been found that the powder pharmaceutical compositions of the present invention are especially effective in the treatment of cold and/or flu symptoms when administered at the composition dosages defined herein at a typical frequency of one to six times per day.

### Method of Manufacture

The powder pharmaceutical compositions of the present invention may be prepared by any known or otherwise effective technique suitable for providing a pharmaceutical composition comprising the pharmaceutical active defined herein, provided that the powder pharmaceutical compositions are dissolvable in a liquid defined herein.

In general, the powder pharmaceutical compositions are prepared by dry mixing the pharmaceutical active, and any optional ingredients such as citric acid and ascorbic acid to form a dry premix. This premix is then continuously mixed with any optional flavoring agents and sweeteners.

The resultant powder pharmaceutical compositions of the present invention are suitable for storage in containers such as pouches, sachets, packets, and the like, prior to the compositions being dissolved in a liquid defined herein. The dissolution of the powder pharmaceutical compositions in the liquid can be accomplished by emptying the composition from the container to form a solution with the liquid. The solution of powder pharmaceutical composition and liquid is preferably administered as a hot liquid solution for the effective relief of cold and/or flu symptoms.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified concentrations are weight-weight percents, unless otherwise specified.

The powder pharmaceutical compositions exemplified hereinbelow in Table 1 are powder dry mixtures that comprise the pharmaceutical active, and any optional ingredients such as flavoring agents and sweeteners. These powder pharmaceutical compositions are then dissolved in a liquid such as hot water for the effective relief of cold and or flu symptoms.

TABLE 1

| | Sample 1 | Sample 2 | Sample 3 | Sample 4 | Sample 5 |
|---|---|---|---|---|---|
| Component | (Wt. %) | (Wt. %) | (Wt. %) | (Wt. %) | (Wt. %) |
| Acetaminophen¹ | 10.00 | 10.00 | 10.00 | 10.00 | 10.00 |
| Phenylephrine | 0.20 | — | 0.20 | 0.20 | — |
| Hydrochloride² | | | | | |
| Ascorbic Acid | 1.20 | 1.20 | 1.20 | 1.20 | 1.20 |
| Citric Acid | 10.50 | 10.50 | 10.50 | 10.50 | 10.50 |
| Honey Buds Flavor³ | 2.00 | 2.00 | 2.00 | 2.00 | 2.00 |
| Honey Powder Flavor⁴ | 2.00 | — | 2.00 | — | 2.00 |
| Natural Lemon Flavor⁵ | 1.20 | — | 1.40 | 1.40 | 1.20 |
| Natural Lime Flavor⁶ | — | 4.00 | — | — | — |
| Sweet-Ung⁷ | 3.40 | — | 3.20 | 3.40 | 3.40 |
| Sodium Saccharin | — | 0.30 | — | — | — |
| Aspartame | — | 0.80 | — | — | — |
| Sugar Extra Fine Granulated | 69.4985 | 71.1985 | 69.4985 | 71.2985 | 69.6985 |
| Dye⁸ | 0.0015 | 0.0015 | 0.0015 | 0.0015 | 0.0015 |
| Total: | 100 | 100 | 100 | 100 | 100 |

Wt. %—weight percent
¹pharmaceutical active available from the Rhodia Incorporation
²pharmaceutical active available from Iwaki Seiyaku Co., LTD/R.W. Greef & Company
³honey buds flavor C&K 315-S available from Complementos Alimenticios S.A. de C.V.
⁴honey powder RT-798-01 flavor available from Givaudan de Mexico S.A. de C.V.
⁵natural lemon FLVP FH4857 flavor available from Ungerer & Company
⁶natural lime flavor, Fries & Fries 212750 available from Fries & Fries
⁷sweet-ung 4601 available from lnnovación Alimentaria S.A. de C.V./Ungerer de Mexico
⁸D&C yellow dye No. 10 available from Warner Jenkinson Company Inc.

What is claimed is:

1. A powder pharmaceutical composition comprising:
(a) a phenylephrine pharmaceutical active;
wherein the composition is dissolvable in a hot liquid.

2. The powder pharmaceutical composition according to claim 1 wherein the composition comprises from about 0.05% to about 0.5% by weight of the phenylephrine pharmaceutical active.

3. The powder pharmaceutical composition according to claim 2 wherein the phenylephrine pharmaceutical active is phenylephrine hydrochloride.

4. The powder pharmaceutical composition according to claim 3 wherein the composition further comprises a pharmaceutical active selected from the group consisting of antitussives, antihistamines, non-sedating antihistamines, expectorants, mucolyptics, analgesics, antipyretics, anti-inflammatory agents, and mixtures thereof.

5. The powder pharmaceutical composition according to claim 4 wherein the analgesics are selected from the group consisting of acetaminophen, aspirin, sodium salicylate, salicylamide, diflunisal, etodolac, fenoprofen, flurbiprofen, ketoprofen, ketorolac, naproxen, and mixtures thereof.

6. The powder pharmaceutical composition according to claim 5 wherein the composition comprises a combination of phenylephrine hydrochloride and acetaminophen pharmaceutical active.

7. The powder pharmaceutical composition according to claim 6 wherein the composition comprises from about 0.08% to about 0.4% by weight of phenylephrine hydrochloride and from about 1% to about 20% by weight of acetaminophen.

8. The powder pharmaceutical composition according to claim 1 wherein the hot liquid is selected from the group consisting of hot water, hot tea, hot milk, hot apple cider, and mixtures thereof.

9. The powder pharmaceutical composition according to claim 8 wherein the hot liquid is hot water.

10. The powder pharmaceutical composition according to claim 1 wherein the composition further comprises a flavoring agent.

11. The powder pharmaceutical composition according to claim 10 wherein the flavoring agent is selected from the group consisting of anise, oil of peppermint, oil of clove, eucalyptus, lemon, lime, honey, honey lemon, red fruit, mint, grapefruit, orange, grape, cherry, cherry cola, berry, and mixtures thereof.

12. The powder pharmaceutical composition according to claim 1 wherein the composition further comprises a sweetener.

13. A method of treating the symptoms of a common cold or flu comprising the steps of:
(a) preparing a powder pharmaceutical composition comprising a phenylephrine pharmaceutical active; and
(b) dissolving the powder pharmaceutical composition of (a) in a hot liquid.

14. The method according to claim 13 wherein the powder pharmaceutical composition further comprises a pharmaceutical active selected from the group consisting of antitussives, antihistamines, non-sedating antihistamines, expectorants, mucolyptics, analgesics, antipyretics, anti-inflammatory agents, and mixtures thereof.

15. The method according to claim 14 wherein the analgesics are selected from the group consisting of acetaminophen, aspirin, sodium salicylate, salicylamide, diflunisal, etodolac, fenoprofen, flurbiprofen, ketoprofen, ketorolac, naproxen, and mixtures thereof.

16. The method according to claim 15 wherein the powder pharmaceutical composition comprises a combination of phenylephrine hydrochloride and acetaminophen pharmaceutical active.

17. The method according to claim 13 wherein the hot liquid is selected from the group consisting of hot water, hot tea, hot milk, hot apple cider, mixtures thereof.

18. The method according to claim 16 wherein the powder pharmaceutical composition further comprises a flavoring agent selected from the group consisting of anise, oil of peppermint, oil of clove, eucalyptus, lemon, lime, honey, honey lemon, red fruit, mint, grapefruit, orange, grape, cherry, cherry cola, berry, and mixtures thereof.

19. The method according to claim 13 wherein the powder pharmaceutical composition further comprises a sweetener.
